(19) United States
(12) Patent Application Publication
(10) Pub. No.: US 2005/0118728 A1
(43) Pub. Date: Jun. 2, 2005

(54) Two-layer antibody capture system

(76) Inventors: Kevin K. Caldwell, Albuquerque, NM (US); Colin T. Buckley, Albuquerque, NM (US)

Correspondence Address: MUETING, RAASCH & GEBHARDT, P.A., P.O. BOX 581415, MINNEAPOLIS, MN, 55458, US

(21) Appl. No.: 10/883,859

(22) Filed: Jul. 2, 2004

Related U.S. Application Data
(60) Provisional application No. 60/484,428, filed on Jul. 2, 2003.

(51) Int. Cl. G01N 33/543

(52) U.S. Cl. 436/518; 435/7.5; 435/7.9

(57) ABSTRACT

A multimolecular complex that includes an anti-immunoglobulin (anti-Ig) linking antibody reversibly bound to a substrate, a primary antibody bound to the anti-Ig linking antibody to yield an immobilized primary antibody, and optionally, an antigen is bound to the immobilized primary antibody; and a method for immunochemically immobilizing a molecule of interest, such as an enzyme, using the multimolecular complex. The binding interaction between the substrate and the linking antibody is preferably a biotin/streptavidin interaction.

This application claims the benefit of U.S. Provisional Application Ser. No. 60/484,428, filed Jul. 2, 2003, which is incorporated herein by reference in its entirety.

### STATEMENT OF GOVERNMENT RIGHTS

This invention was made with government support under a grant from the National Institutes of Health, Grant No. AA12635 and the U.S. Department of Army, Grant No. DAMD17-00-1-0582. The U.S. Government has certain rights in this invention.

### BACKGROUND OF THE INVENTION

Current approaches used for the separation of a biological component such as an enzyme from a complex mixture (e.g., cell or tissue extract) employ chromatographic, electrophoretic or immunologic methods. Compared to chromatographic and electrophoretic techniques, immunologic techniques are often easier, may allow for increased yields, and are applicable to the processing of a wide range of sample sizes. Antibodies which are capable of separating a native (as opposed to denatured) form of an enzyme would be desirable as they would allow for in vitro measurements of enzyme activity.

### SUMMARY OF THE INVENTION

The invention provides a multimolecular complex that includes an anti-immunoglobulin (anti-Ig) linking antibody bound to a substrate. Preferably, the binding interaction between the linking antibody and the substrate is reversible. A primary antibody is bound to the anti-Ig linking antibody to yield an immobilized primary antibody. Optionally, an antigen is bound to the primary antibody.

In a preferred embodiment, the reversible binding between the substrate and the linking antibody is achieved using a biotin/streptavidin interaction, although other reversible linkages are contemplated as well. For example, a preferred multimolecular complex of the invention includes a substrate-bound streptavidin, and a biotinylated anti-immunoglobulin (anti-Ig) linking antibody bound to the streptavidin. In another preferred embodiment, the invention includes a substrate-bound biotin and a streptavidin-labeled anti-immunoglobulin (anti-Ig) linking antibody bound to the biotin.

In alternative embodiments, the linking antibody comprises a modified anti-immunoglobulin antibody comprising a biotinylated F_{AB} fragment. In other alternative embodiments, the primary antibody comprises a modified antibody comprising a fusion between an F_{C} fragment and a receptor, and the multimolecular complex is used to detect the receptor ligand instead of an antigen.

If the multimolecular complex includes a bound antigen, the bound antigen is preferably a biomolecule; more preferably it is an enzyme. The enzyme may be biologically active or inactive when bound to the primary antibody, although the invention is particularly well suited for the immobilization of biologically active enzymes. The bound antigen can include, or be a part of, a cell or a protein.

In one embodiment, the multimolecular complex includes a second primary antibody which is bound to a site on the antigen, particularly a cell or protein, that is different from the site bound to the immobilized primary antibody. The second primary antibody is preferably in solution (i.e., not immobilized) prior to binding the antigen, and, particularly if it is detectably labeled, may be useful in detecting the bound antigen, for example in a sandwich assay. Preferably, the second primary antibody binds to a site on the antigen that is different from the site bound to the immobilized primary antibody.

Also provided is a method for immunochemically immobilizing a molecule of interest, such as an enzyme, using the multimolecular complex of the invention. For example, a biotinylated anti-immunoglobulin (Ig) bound to a streptavidin-coated substrate can be used to immobilize primary antibodies that bind an antigen of interest, which, in turn, are used to capture the antigen from a sample, thereby immobilizing the immune-complex on the solid-phase support. The sample can be, for example, a biological sample, an environmental sample, a food sample, or a cosmetic sample.

Optionally, the method further includes disrupting the binding interaction between the immobilized primary antibody and the anti-Ig linking antibody so as to dissociate the primary antibody from the anti-Ig linking antibody. Alternatively or additionally, the method optionally further includes disrupting the binding interaction between the antigen and the primary antibody so as to dissociate the antigen from the primary antibody.

If desired, the bound ligand can be quantified. If the bound antigen is an enzyme, the method further optionally includes assaying the bound enzyme for biological activity. The bound enzyme can be active or inactive.

In embodiments of the invention wherein the multimolecular complex includes a bound, biologically active enzyme, the invention further includes a method for contacting the bound enzyme with an enzyme substrate to cause an enzymatic reaction. This embodiment for the invention is well-suited to industrial processes.

In embodiments of the invention wherein the multimolecular complex includes a bound cell or protein antigen, the method of the invention optionally includes sorting or purifying the antigen.

The invention also includes a method for detecting the presence of an antigen in a sample. The method involves contacting the multimolecular complex of the invention with a sample comprising an antigen, to yield a multimolecular complex comprising an antigen bound to the immobilized primary antibody, followed by detecting the presence of the bound antigen. The bound antigen can be detected immunologically, using a sandwich assay such as an enzyme linked immunosorbant assay (ELISA). Optionally, the bound antigen is contacted with a second primary antibody that binds an epitope on the bound antigen that differs from the epitope bound by the immobilized primary antibody. The second primary antibody is preferably detectable or detectably labeled. The bound antigen can be detected using immunologic, spectroscopic, thermodynamic or kinetic methods, for example.

In embodiments in which the bound antigen is an enzyme, the bound enzyme can be assayed for biological activity. In a preferred embodiment, the presence or absence of the bound enzyme is detected using an activity assay together with an immunological assay; wherein success in detecting the bound enzyme immunologically combined with failure to detect bound enzyme activity is indicative of the binding of a non-active form of the enzyme to the immobilized primary antibody.

An antigen can be detected in a biological sample, an environmental sample, a food sample, a cosmetic sample or pharmaceutical sample, for example. The method of the invention is particularly useful when the antigen is a contaminant, and wherein the method is performed for quality control purposes.

### BRIEF DESCRIPTION OF THE DRAWINGS

FIG. 1 is a schematic drawing of an embodiment of the two-layer antibody system of the invention showing the capture and detection of an antigen in an enzyme-linked immunosorbant assay (ELISA). (A) In this embodiment, the primary (capture) antibody is immobilized onto the streptavidin-coated solid phase through the use of biotinylated anti-immunoglobulins antibodies. The antigen of interest is bound by the primary (capture) antibody. (B) The captured antigen can be detected by a variety of means. In this embodiment the antigen is labeled with a second, primary antibody. (C) The labeled antibody in this embodiment can then be detected with a conventional colorimetric assay using horseradish peroxidase-labeled anti-immunoglobulin secondary antibody that binds the second, primary antibody.

FIG. 2 is a schematic drawing of an embodiment of the two-layer antibody system of the invention showing capture of an antigen, which in this illustration is (A) a biomolecule in solution or (B) a marker on the surface of a cell, using streptavidin-coated beads. A second antibody can be used for detection of the captured antigen. The second antibody can bind to the captured biomolecule as shown in (A), to the cell marker of the captured cell, or to a different cell marker on the surface of the captured cell as shown in (B). In (C), contacting the resulting multimolecular complex with free immunoglobulin to which the anti-Ig linking antibody binds (isotype match) is shown to release the antigen, which in this illustration is present on the surface of a cell. The antigen (in this illustration, still attached to the surface of the cell), which is no longer immobilized, remains bound to the primary (capture) antibody.

FIG. 3 is a schematic drawing of an embodiment of the two-layer antibody system of the invention showing capture and detection of an enzyme antigen. The primary (capture) antibody is immobilized onto the streptavidin-coated solid phase using a biotinylated anti-immunoglobulin antibody. The antigen of interest is bound by the primary (capture) antibody. The captured antigen possesses catalytic activity making it possible to detect the antigen by measuring the formation of the enzyme reaction product.

FIG. 4 is a schematic drawing of an embodiment of the two-layer antibody system of the invention showing a modified primary antibody formed from a F_{C} region fused to a receptor protein. The receptor captures its ligand, analogous to the capture of an antigen with the variable region (F_{AB}) of a primary antibody.

FIG. 5 is a schematic drawing showing phospholipase C-γl (PLC-γl) immobilization and activity assay using biotinylated goat anti-rabbit IgG and streptavidin coated microtiter wells according to the invention.

FIG. 6 shows a determination of the quantity of rabbit anti-phospholipase C-γl antibody that can be adhered to the surface of biotinylated anti-rabbit IgG coated streptavidin plates.

FIG. 7 shows the time course of phospholipase C-γl activity captured from rat brain S1 fraction.

FIG. 8 shows determination of the phospholipase C-γl activity captured from increasing tissue concentrations.

FIG. 9 demonstrates that the tyrosine kinase inhibitor, genistein, blocks ATP-dependent stimulation of hippocampal formation P2 fraction phospholipase C-γl catalytic activity.

### DETAILED DESCRIPTION OF THE PREFERRED EMBODIMENTS

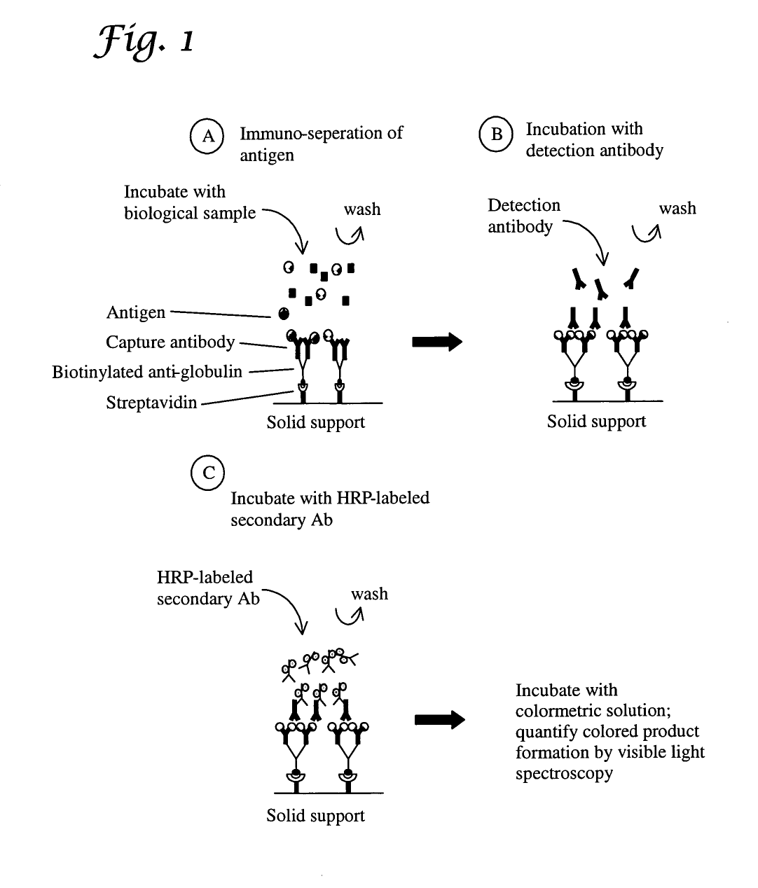

The present invention is directed to a multimolecular complex that includes, for example, (i) a substrate-bound streptavidin; (ii) a biotin-labeled (“biotinylated”) anti-immunoglobulin (anti-Ig) linking antibody bound to the streptavidin via a binding interaction between the streptavidin and the biotin; and (iii) a primary (“capture”) antibody bound to the anti-Ig linking antibody via a binding interaction between the variable region of the anti-Ig linking antibody and the constant region of the primary antibody. The invention is exemplified in FIGS. 1-3.

This multimolecular complex is referred to herein as a “two-layer” system, the first layer being formed from the linking antibody, and the second layer being formed from the primary antibody. Advantageously, the invention allows for capture or immobilization of an antigen at a distance from the substrate surface, since a linking antibody is interposed between the primary antibody/antigen complex and the substrate surface. Optionally, the multimolecular complex of the invention further includes an antigen bound to the variable region of the primary antibody.

In an alternative embodiment of the invention, roles of the streptavidin and biotin are reversed, with the biotin being substrate-bound and the streptavidin being bound to the linking antibody. However, the embodiment that includes the biotinylated linking antibody is preferred since many biotinylated linking antibodies are readily available commercially.

It should further be understood that the molecular linkage that connects the substrate and the linking antibody is not, in any event, limited to a streptavidin-biotin interaction. For example, a chelator (e.g., a metal chelator) can be linked to a linking antibody, which linking antibody can then bind to an ionic surface. After contact with the antigen, the antibody-antigen complex can be dissociated from the surface using an ion that competes with the ionic surface for binding to the chelator. Another alternative is to use a histidine tagged linking antibody, which would allow for attaching the linking antibodies to nickel-coated surfaces. Other molecular linkages that can connect the substrate to the linking antibody could include protein/protein interactions, hydrophobic interactions, nucleic acid binding interactions such as hybridization, and RNA aptamer interactions. In preferred embodiments the linkage between the substrate and the linking antibody is reversible to allow dissociation of the linking antibody-primary antibody-antigen (if present) subcomplex from the substrate, if desired.

The linking and primary antibodies used in the multimolecular complex of the invention may be, independently, monoclonal or polyclonal antibodies. They can be made using any convenient method known to the art, such as production in an animal, from a hybridoma, or from a phage display library (de StGroth et al., Immunol. Meth., 1980;35(1-2)1-21; Kohler et al., Nature, 256 (1975) 495-7; Current Protocols in Immunology (2001) Wiley Interscience). The antibodies are preferably vertebrate antibodies; more preferably they are avian or mammalian antibodies.

Anti-Ig linking antibodies bind to the constant region of a primary antibody and can include anti-cat, -chicken, -cow, -dog, -goat, -guinea pig, -hamster, -horse, -human, -mouse, -rabbit, -rat, -sheep and -swine antibodies, preferably anti-Ig antibodies. Also useful as linking antibodies are modified antibodies such as biotinylated F_{AB} fragments that bind antibodies, as described in more detail below.

Primary and/or linking antibodies are preferably cat, chicken, cow, dog, goat, guinea pig, hamster, horse, human, mouse, rabbit, rat, sheep or swine antibodies. Modified antibodies, such as humanized antibodies, can also be utilized.

The antibodies used in the invention are preferably IgG antibodies but can include any class of antibodies including IgE, IgM, IgA, IgD, IgY and the like.

The linking and primary antibodies are independently selected, subject to the constraint that the linking antibody binds the primary antibody. For example, one antibody may be a polyclonal antibody, while the other is a monoclonal. As another example, the linking antibody may be a biotinylated rabbit IgG antibody that binds to a human IgA (i.e., a rabbit anti-human IgA gamma immunoglobulin) and the primary antibody may be a human IgA antibody. Notably, the antigen immobilization system of the present invention is based on the selectivity of the primary antibody. This allows the use of a second primary antibody (in solution) in a sandwich assay (as described in more detail below) that is not of the same type as the primary antibody in the assay. For example, a biotinylated goat anti-rabbit antibody (linking antibody) could be used to immobilize a rabbit IgG (primary antibody) onto the streptavidin-coated surface; the rabbit antibody captures an antigen of interest, and a second primary antibody, such as a mouse IgG, raised against the same antigen could be used to label that antigen of interest. The invention is in no way limited by the type of antibodies used, as long as the linking antibody is capable of binding to the primary antibody of interest to create the multimolecular complex of the invention.

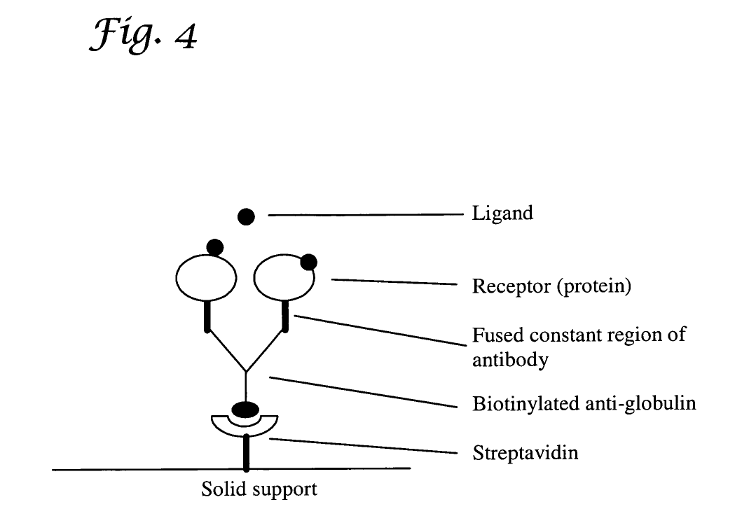

The linking antibody binds the constant region (F_{C}) of the primary antibody. The variable region of the primary antibody (F_{AB}) can be substituted with a receptor molecule or other protein which forms a fusion with the F_{C} region recognized by the linking antibody. Receptor binding to a ligand in this modified antibody is analogous to the variable region binding to an antigen (FIG. 4). In this way, the receptor ligand can be captured from solution. The ligand can be detected, for example, by ELISA as described herein. Capture of the ligand can also be used to remove an undesirable ligand from a medium, such as from circulation in the blood. Examples of F_{C} fusions include fusions to a tumor necrosis factor alpha receptor or a major histocompatibility receptor, fused to the constant region of antibodies could be adhered to any of the surfaces described in this document. See Lev et al., Proc. Natl. Acad. Sci. USA 101 (2004) 9051-9056; Masteller et al., J. Immunol. 171 (2003) 5587-5595; Olsen et al., New Engl. J. Med. 350 (2004) 2167-2179.

Likewise, modified linking antibodies such as biotinylated F_{AB} fragments that bind primary (capture) antibodies could be used in the assay.

The antigen to which the primary antibody of the multimolecular complex is capable of binding can be any selected molecule. Preferably, the antigen is a biomolecule such as a polypeptide, a polynucleotide, a carbohydrate, a lipid, a hormone, a metabolite, a natural product and the like. The term “polypeptide” refers to a linear chain of amino acids and includes a peptide, oligopeptide and protein. It is to be understood that the invention is not limited by the length or the function of the polypeptide detected. The term “peptide” may be used to connote a shorter polypeptide such as dipeptide, tripeptide, or oligopeptide, typically connoting a polypeptide having between 2 and about 50 amino acids. The term “protein” is often applied to longer polypeptides and includes a folded polypeptide of any length having structural, enzymatic or other active properties, such as secondary or tertiary structure, distinct domains or hydrophobic cores, catalytic activity and the like. Regardless of the nomenclature used, however, no limitations on the length or the function of the polypeptide antigen that is detectable according to the invention are intended.

In a preferred embodiment, the antigen to which the primary antibody is capable of binding is a protein, more preferably an enzyme. Advantageously, the invention permits selective detection and in vitro characterization of a wide variety of enzyme isoforms. In embodiments of the multimolecular complex of the invention that include, or are used to detect, a protein antigen, the protein antigen, such as an enzyme, can be active or it can be inactive. A multimolecular complex that includes a bound protein antigen may exhibit full activity, reduced activity, or no detectable activity.

In another preferred embodiment, the antigen to which the primary antibody is capable of binding is present on the surface of a cell. This allows cells to be sorted and purified using the method of the invention. As discussed earlier, the bound cell (or cellular component) can be detected using a second primary antibody that binds to another antigen that is present on the surface of the cell, such as a protein, lipid, carbohydrate or the like.

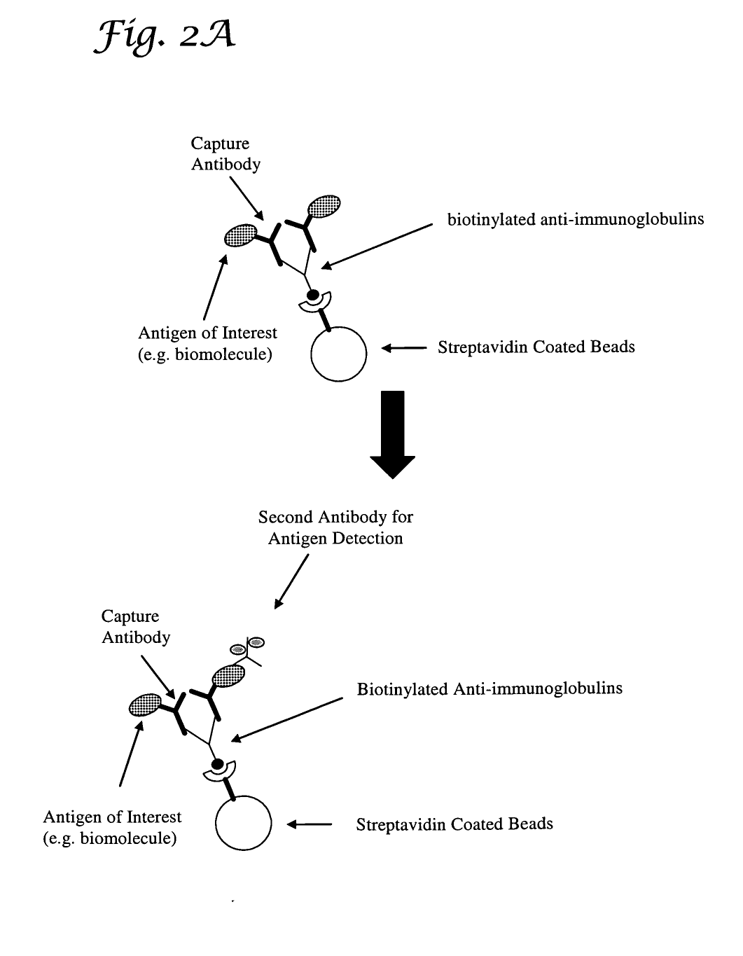

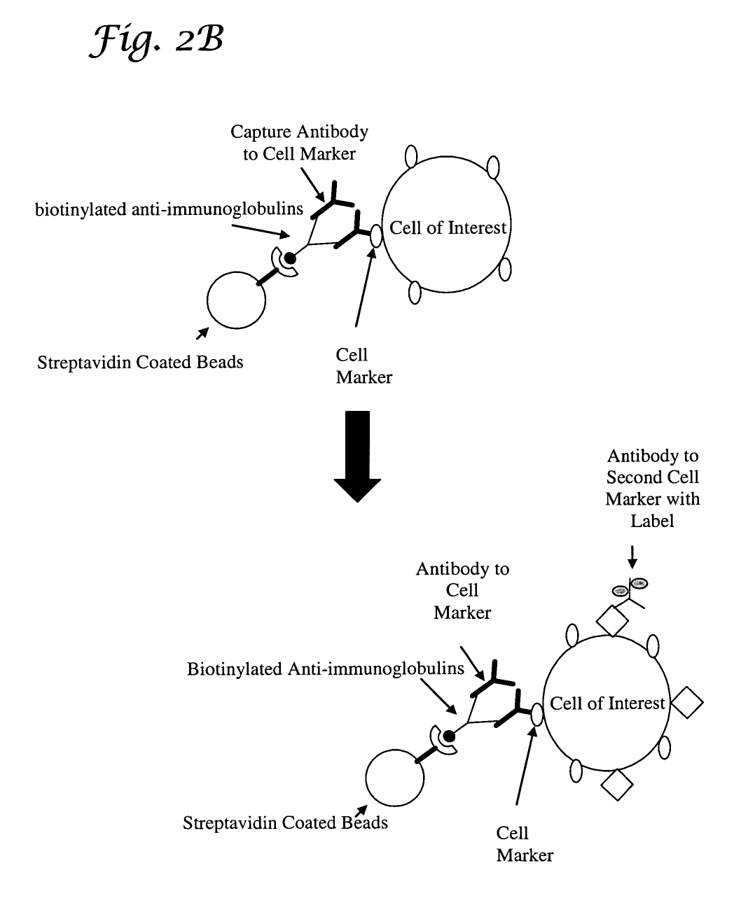

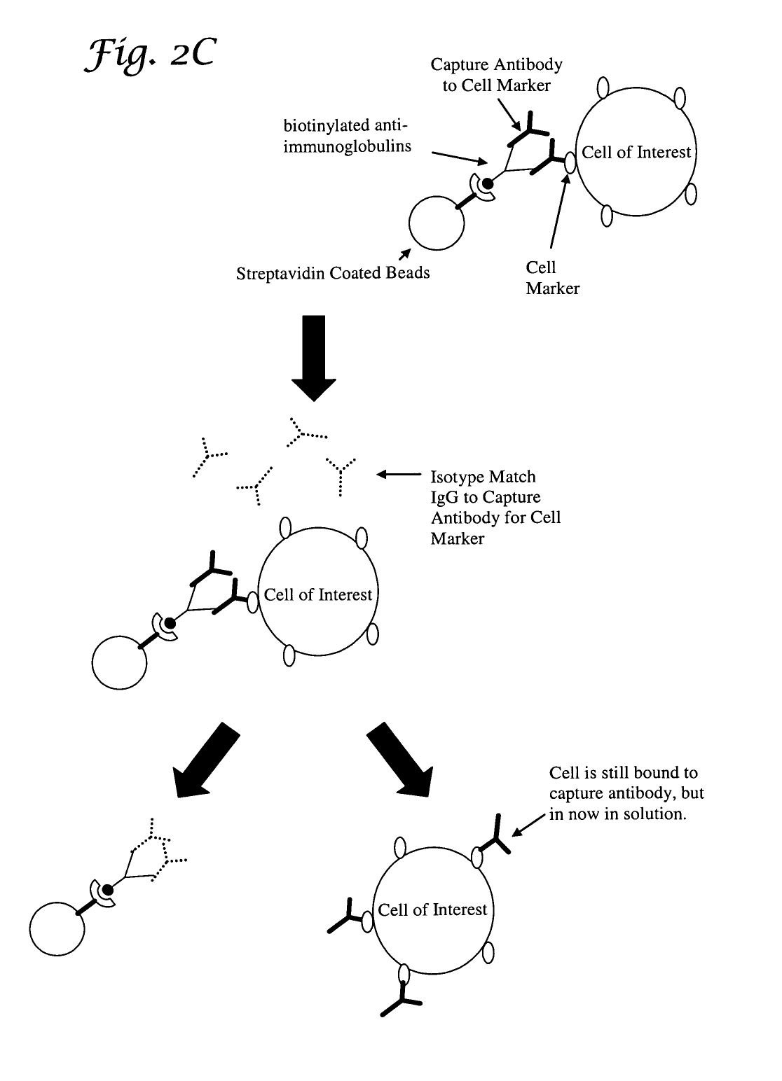

The linking antibody in the multimolecular complex of the invention is bound to a substrate. It should be understood that the term “substrate” in this context means a stationary surface to which the multimolecular complex can attach. This meaning of “substrate” should not be confused with the meaning of “substrate” as it is used in enzymology to refer to a specific molecule upon which an enzyme acts. For example, binding of streptavidin to the substrate occurs at a surface of the substrate so that it is able to contact, and bind with, the biotin moiety of a biotinylated linking antibody. The substrate can take the form of an immobilized surface, such as a membrane, the bottom of a well on a microtiter plate or a position on a microchip. The technique can also be expanded to include other solid phase supports that are coated with streptavidin, such as streptavidin-coated beads and streptavidin-coated microfuge tubes. The immobilization of antibodies onto a solid phase support of streptavidin coated beads is shown in FIG. 2. Optionally, the beads can include a magnetic form, thereby greatly facilitating cell sorting and separation. In this method, the biotinylated anti-immunoglobulins antibodies are attached to the streptavidin coated beads and then used to immobilize the antibody to the antigen of interest, such as a cell or protein. A second primary antibody is then used to label a second marker which labels the cell or protein at a different site.

Streptavidin-bound substrates can be custom-made, or they can be readily obtained commercially, for example from Roche (Basel, Switzerland). Materials suitable for use as substrates include polymeric materials and plastics, particularly organic polymers; silica-based substrates such as glass, quartz, silicon and polysilicon including silicon wafer; ceramic; metals; beads (porous or non-porous) of cross-linked polymers (e.g., dextran, agarose, etc.); composite materials; and the like. Optionally the substrate is coated with a material, for example, gold, titanium oxide, silicon oxide, etc. that allows derivatization of the surface.

As noted above the substrate can take the form of a planar surface, a well, a microchip, a bead, and so on. It should be understood that the invention is not limited by the material or form of the substrate, which can be selected to fit the application of interest to the researcher.

The multimolecular complex of the invention lends itself to many useful and varied applications, as it has a number of advantages over art-recognized methods for antigen capture that will be described below. Some applications are exemplified here, but many more will be readily apparent to one of skill in the art as the invention is broadly applicable across the entire art areas of molecular biology, biochemistry, immunology and related fields.

The invention thus broadly includes a method for immobilizing or capturing a selected antigen. A sample suspected of containing an antigen of interest is contacted with a multimolecular complex of the invention that contains a primary antibody that binds the selected antigen. The resulting product is a multimolecular complex that now includes the bound antigen.

In a preferred embodiment, the multimolecular complex, working through the primary antibody, is used to capture an antigen, typically a biomolecule of interest, from a biological sample. Examples of biological samples that can be used in the method of the invention include bodily tissues and fluids, such as tissue sections, blood, serum, and plasma, as well as secretions or excretions such as mucus, tears, saliva, urine, vaginal and rectal secretions, and the like. The biological sample can be a crude, unpurified sample, or it can be partially fractionated. Optionally, the biological sample can be treated, for example with one or more proteases, nucleases, surfactants and the like, prior to contact with the multimolecular complex of the invention. The invention is well suited for medical and veterinary applications.

In another preferred embodiment, the sample to be analyzed is an environmental sample, such as water or a solubilized soil sample. In yet another preferred embodiment, the sample is a food or other sample prepared in the course of manufacturing or processing a food, and the method of the invention is used to maintain control over the quality or safety of a food product. In various quality control applications, the method of the invention can be used to assess the level of a contaminant antigen in any sample of interest, such as a pharmaceutical sample, cosmetic sample and the like. In general, the method of the invention can be applied to any embodiment wherein the antigen is solubilized or extracted, if necessary, and presented to the macromolecular complex in a liquid or semi-liquid sample.

In a particularly preferred embodiment, the method of the invention is used to capture an enzyme antigen from a biological sample, typically, but not limited to, a body fluid, cell extract or supernatant, or solubilized tissue sample. The invention is especially well-suited to capturing or immobilizing enzymes because, contrary to other methods known to the art, the bound enzyme in the multimolecular complex of the invention often remains active.

The failure of prior art methods to preserve the activity of the bound enzyme can be traced, at least in part, to various challenges that have been reported for immobilizing antibodies onto surfaces. The main problem resides with the types of surfaces used in the art to immobilize the antibody. These surfaces tend to use strong, nonspecific protein adsorption via ionic interactions, van der Waals forces, and polar-polar interactions for adsorption of the antibody to the surface (Lin et al., J. Immunol. Methods 125 (1989) 67-77). If these interactions are strong enough, the primary antibody and/or the protein antigen may be completely or partially denatured by the surface (Yakovleva et al., Anal. Chem. 74 (2002) 2994-3004).

The linkage used in the present method avoids these issues by using a molecular linkage, such as a streptavidin-biotin interaction, to position a linking antibody away from the surface. Distancing the primary antibody from the surface in accordance with the invention reduces the likelihood that the primary antibody is subjected to the denaturing forces described above. Additionally, there is a greater likelihood that a captured enzyme will retain its catalytic activity because it is sufficiently removed from the surface of the substrate to avoid denaturation of the enzyme by the surface.

Another important advantage is that the present method avoids the necessity of biotinylating the primary (capture) antibody. The biotinylation step is applied to the linking anti-globulin, so that the primary antibody remains unaffected by the biotinylation process. Biotinylated anti-globulins are a common reagent used to identify primary antibodies bound to antigens in immunohistochemistry and Western blotting. Thus, biotinylated antibodies are commercially available and have been developed with specificity for immunoglobulins, allowing for the application of this procedure for immobilizing a variety of antibodies from different species and different immunoglobulin isotypes quickly and uniformly. This reduces both time and assay costs.

The presence of a bound antigen can be detected in any convenient way. For example, if the antigen is an enzyme, the bound complex can be assayed for the presence of enzyme activity, using any known method for assaying the particular activity of the enzyme. Another example is the use of a sandwich assay, such as an enzyme linked immunosorbant assay (ELISA), to detect the bound enzyme. The bound antigen is contacted with a second primary antibody (in solution) that binds an epitope on the antigen that differs from the epitope bound by the immobilized primary (capture) antibody. Optionally, the second primary antibody is labeled, for example with horseradish peroxidase, to facilitate detection of the antigen using methods well known to the art. An ELISA assay for detecting the bound antigen in the multimolecular complex of the invention is shown in FIG. 2. In this figure the capture antibody is immobilized onto the solid phase coated with streptavidin by biotinylated anti-immunoglobulins antibodies. The antigen of interest is captured, and then labeled with a second antibody that can be detected with a conventional calorimetric assay using horse-radish peroxidase or other types of known detection methods.

In some embodiments wherein the antigen is an enzyme, detection based on activity as well as immunoassay, such as ELISA, can be advantageously combined. This combination permits the study of both the enzyme, in terms of its catalytic activity and concentration, as well as the antibody used for the capture step. For example, when a primary antibody is raised against a single determinant, such as a linear peptide corresponding to a portion of the enzyme of interest, the antibody may or may not recognize the native three-dimensional structure of the enzyme. To test whether it recognizes the native structure of the enzyme, the primary antibody can be adhered to the surface as described above, the enzyme can be captured, the enzyme's substrate can be applied, and the product formation can be detected using a conventional method to measure the product. The formation of product suggests the antibody is capable of recognizing the enzyme's native structure and capturing that enzyme in a catalytically active form. Alternatively, the antibody could capture the enzyme, but the enzyme may not be enzymatically active. The activity assay would not show enzymatic product formation; however, the immunoassay could then be used to determine if the enzyme were captured, albeit in an inactive state. Preferably, the activity of the enzyme is first assayed, then the amount of bound enzyme is quantitated, for example by immunoassay, in case the quantitation process might impair enzyme activity.

Other ways of detecting the binding of the antigen make use of changes in energy upon binding. Calorimetric methods, such as differential scanning calorimetry, can be used, for example. Binding of an antigen may also be detected using spectroscopic methods such as fluorescence, UV and infrared spectroscopy, Raman spectroscopy, circular dichroism, conventional electrochemistry employing electrodes suitable for the particular application, and the like.

Advantageously, the bound antigen can be removed from the biotinylated anti-immunoglobulins antibody surface by flooding the substrate surface with the same type of antibody as the primary antibody, thereby removing the bound antigen from the surface. The antigen will still be bound to the primary (capture) antibody. This allows the antigen, which at that point is still attached to the primary antibody, to be put back in solution for further analysis. The reversibility of the binding interaction between the linking antibody and the primary antibody is important for the recovery of certain antigens, such as cells or enzymes, whose analysis is enhanced in solution.

Optionally, the antigen can also be separated from the primary antibody by, for example, changing the pH of the eluant. For example, a glycine wash may be used to dissociate the antigen from the primary antibody. This can be done in lieu of detaching the primary antibody from the linking antibody, or either before or after detaching the primary antibody from the linking antibody.

The ability to capture active enzymes from tissues using the method of the present invention has strong human diagnostic implications. For example, one complicating factor for the analysis of human serum is that this serum contains human IgG. This complicating factor essentially precludes the use of antibody binding proteins, such as protein A or protein G, to immobilize capture antibodies for clinically important analytes to solid phases due to the background interference of the human IgG. To address this issue, prior art methods utilize biotinylated antibody against the analyte of interest, or direct adsorption of the capture antibody onto a surface. However, both of these prior art approaches have serious limitations. As noted above, biotinylation of multiple capture antibodies for multiple antigens is expensive and time consuming. In addition, as noted above, the biotinylated capture antibody may be close enough in proximity to the surface to allow for the surface to denature the bound enzyme or protein of interest. Furthermore, the biotinylation procedure can render the antibody less effective at capturing its antigen. The direct adsorption of the antibody to the surface may render the antibody incapable of binding to the protein or enzyme of interest, requires large amounts of the primary antibody in the coating conditions, and also has the limitation of placing the captured protein in proximity to the solid phase. By utilizing the method of the present invention, these limitations can be avoided, and the presence of human IgG in the biological sample can be accommodated.

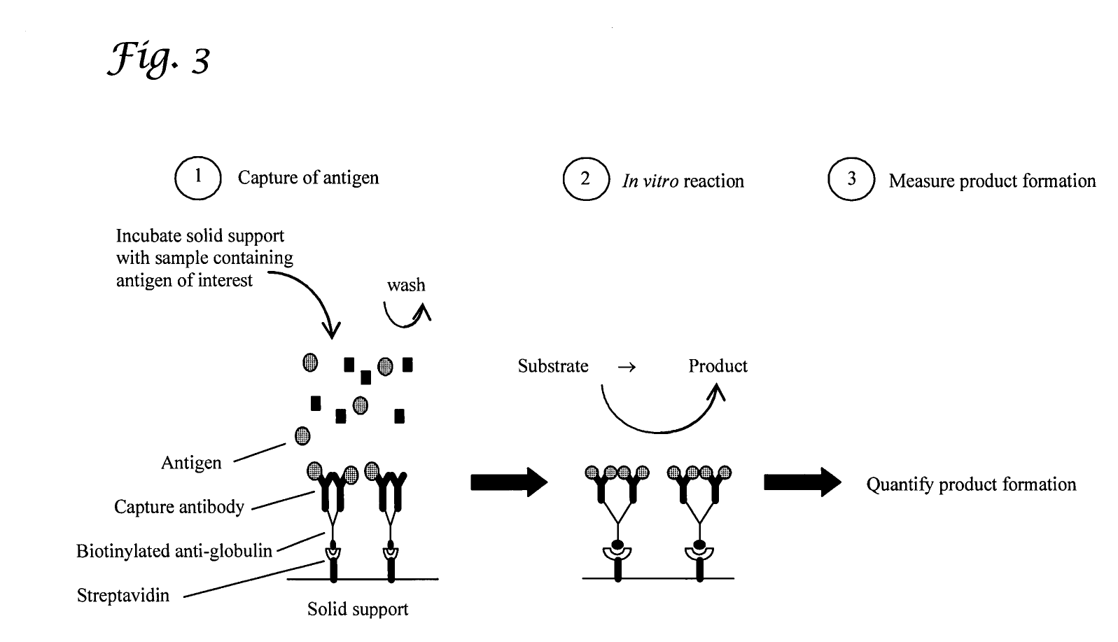

Multimolecular complexes of the invention that include a bound enzyme antigen have many scientific and medical uses. For example, the bound, active enzyme can be used for carrying out chemical or enzymatic reactions, e.g., in series or parallel, in a channel, or in a flow cell. An enzyme of interest (or other molecule) can be affinity purified using a multimolecular complex of the invention as the affinity substrate, and its activity can be assessed while it is still bound to the affinity substrate (FIG. 3). A biological sample can be detoxified by the removal of a contaminant that binds to a multimolecular complex of the invention. The bound, active enzyme can be used to screen candidate compounds for their utility as enzyme inhibitors. The method is applicable to the high throughput screening of regulators (e.g., inhibitors) of enzyme activity, antibodies, and the identification of interacting proteins. The uses of the multimolecular complex of the invention are limited only by the creativity of the researcher.

A potentially significant application of the method of the invention for analyzing antigens, particularly enzyme antigens, is its suitability for immobilizing an antigen to a microfluidic chip-based device. Currently, a common method for immobilizing an enzyme on a chip is to use a biotin-streptavidin enzyme adhered to a streptavidin/biotin-coated surface. Several drawbacks are associated with this approach. The enzyme must first be biotinylated or coated with streptavidin prior to being adhered to the surface. This biotinylation/streptavidin coating step may irreversibly modify (e.g., alter the catalytic functions of) the enzyme. By using antibodies according to the present invention, and in particular those that do not interfere with the enzyme's catalytic activity, it is possible to avoid some of the issues described in the current literature.

Another application of the present invention involves protein characterization. Protein characterization is commonly performed using antibody-based techniques to isolate the protein of interest and study its biological function. The properties of the antibody are important in these types of analyses. For example, antibodies that recognize a protein's native structure can capture the protein of interest onto a solid support, such as protein A beads or other surfaces, and may allow for determination of the protein's biological function, such as enzymatic activity. Antibodies that bind to a linear peptide sequence, on the other hand, can still bind to the protein under denaturing conditions, but may not allow for analysis of biological function. Furthermore, in order to study the protein's function, such as enzymatic activity, it is important that the antibody not bind to the enzyme in a manner that interferes with the protein of interest's biological function. However, antibodies that do inhibit enzyme activity or protein function are also important because these antibodies have potential for disrupting the function in biological systems, which may be of scientific or therapeutic interest. It is, therefore, important to identify antibodies that are capable of capturing proteins that retain biological functions, as well as those antibodies that capture the protein and disrupt the biological function of the captured protein. In addition, it is important to create techniques or methods that allow for distinguishing which antibodies perform the function described above. Ideally, these methods must be easy to perform, inexpensive compared to common techniques, and applicable to any antibody available for use. The method of the invention is ideally suited for these types of protein analyses.

The present invention is illustrated by the following examples. It is to be understood that the particular examples, materials, amounts, and procedures are to be interpreted broadly in accordance with the scope and spirit of the invention as set forth herein.

### EXAMPLES

The following examples demonstrate the validity of the method for the study of one of the primary phosphatidylinositol-specific phospholipase C isozymes, phospholipase C-γl, found in rodent brain. Phospholipase C isozymes catalyze the hydrolysis of phosphatidylinositol 4,5-bisphosphate (PtdIns(4,5)P₂)¹, yielding inositol 1,4,5-trisphosphate (Ins(1,4,5)P₃) and 1,2-diacylglycerol (Majerus et al., Cell 63 (1990) 459-465; Williams, Biochim. Biophys. Acta 1441 (1999) 255-267). Complementary DNA clones have been isolated for 11 distinct mammalian phospholipase C isozymes (Rhee et al., Annu. Rev. Biochem. 70 (2001) 281-312). Comparison of the predicted amino acid sequences of these clones reveals that phospholipase C isozymes may be grouped into four types: phospholipase C-β, -δ, -γ, and -ε. Most, if not all, of these phospholipase C isozymes are present in brain (Watanabe et al., Eur. J. Neurosci. 10 (1998) 2016-2025; Homma et al., Biochem. Biophys. Res. Commun. 164 (1989) 406-412; Lee et al., J. Biol. Chem. 271 (1996) 25-31; Kelley et al., EMBO J. 20 (2001) 743-754), making studies of the individual isoforms present in this tissue difficult.
¹ PtdIns(4,5)P₂, phosphatidylinositol 4,5-bisphosphate; Ins(1,4,5)P₃, inositol 1,4,5-trisphosphate; PBS, phosphate-buffered serum

Phosphorylation has been shown to influence the catalytic activity of a variety of enzymes, including phospholipase C-γ and -β isozymes. Several studies have demonstrated that phospholipase C-γ catalytic activity is regulated by tyrosine phosphorylation (Rhee et al., Annu. Rev. Biochem. 70 (2001) 281-312; Wahl et al., J. Biol. Chem. 267 (1992) 10447-10456). We demonstrate that the study of the role of protein kinase-dependent regulation of phospholipase C-γl is possible employing the method that we describe for the immobilization of the enzyme on microtiter plates.

### Example I

### Two-Layer Antibody Capture of Enzymes on the Surface of Microtiter Plates: Application to the Study of the Regulation of Phospholipase C-γl Catalytic Activity

Materials and Methods

Materials

Rabbit polyclonal antibodies against phospholipase C-γl, biotin conjugated goat anti-rabbit IgG and normal rabbit IgG were purchased from Santa Cruz Biotechnology (Santa Cruz, Calif.). Aprotinin, [4-(2-Aminoethyl)benzenesulfonylfluroide], leupeptin, calpain III, and genistein were from Calbiochem (San Diego, Calif.). Sodium vanadate was purchased from Fisher Chemical (Fair Lawn, N.J.). Immunoware microtubes and bovine serum albumin, fraction V, were purchased from Pierce (Rockford, Ill.). Dulbecco's phosphate-buffered saline (PBS) was obtained from Biowhittaker (Walkersville, Md.). Triton X-100, PtdIns(4,5)P₂, and Streptawells®, streptavidin coated microtiter plates, were from Roche (Indianapolis, Ind.). [³H]PtdIns(4,5)P₂ was obtained from Perkin-Elmer (Boston, Mass.). Assay Dilution Buffer I, Kinase Inhibitor Cocktail, and Magnesium/ATP Cocktail were from Upstate Biotechnology (Lake Placid, N.Y.).

Animals

All procedures involving rats were approved by the University of New Mexico Health Sciences Center Laboratory Animal Care and Use Committee. Four- to seven-months-old female Sprague-Dawley rats (Harlan Industries, Indianapolis, Ind.) were housed in a constant (22° C.) temperature room on a 16 hour dark/8 hour light schedule (lights off from 1730 to 0930 hours). All rats were provided ad libitum access to standard rat chow and tap water.

Preparation of Triton X-100-Soluble Extracts from Rat Brain

Brains from five rats were removed and homogenized as described in Weeber et al. (Neurobiology of Learning and Memory 76 (2001) 151-182) except the volume of homogenization buffer (20 mM Tris-HCl, pH 7.4, 1 mM EDTA, 320 mM sucrose, 40 μg leupeptin/mL, 20 μg aprotinin/mL, 30 μM calpain III inhibitor, 0.5 mM [4-(2-Aminoethyl)benzenesulfonylfluroide], and 200 μM sodium orthovanadate) was 45 mL. Homogenates were centrifuged (1,000×gₘₐₓ, 10 minutes, 4° C.) to separate crude soluble (S1) and particulate (P1) fractions. The supernatant was decanted and stored in ice. The pellet was resuspended in 10 mL of homogenization buffer, homogenized, and spun as before. The supernatant was decanted, combined with the supernatant from above to form the S1 fraction, and brought to 16 mM (1.0% v/v) Triton X-100, 75 mM KCl and 75 mM NaCl. The mixture was left at 4° C. for 20 minutes, then centrifuged (20,000×gₘₐₓ, 20 minutes, 4° C.) to remove the Triton X-100-insoluble material. The Triton X-100-soluble material was collected, rapidly frozen in liquid nitrogen and stored at −80° C. until further analysis. The total protein concentration of the sample was determined by the method of Bradford (Anal. Biochem. 72 (1976) 248-254), using the BioRad (Richmond, Calif.) protein assay kit; bovine serum albumin served as the protein standard for these determinations.

Tissue Preparation and Subcellular Fractionation of Rat Hippocampus

The preparation of the Triton X-100 extract of the 200,000×gₘₐₓ, postnuclear particulate (P2) preparation derived from rat hippocampal formation was performed essentially as described in Weeber et al. (Neurobiology of Learning and Memory 76 (2001) 151-182). Briefly, the hippocampal formation was removed and homogenized as described. Homogenates were frozen in liquid nitrogen, and stored at −80° C. until further fractionated. Frozen rat hippocampal tissue homogenates were thawed on ice, then centrifuged, (1,000×gₘₐₓ, 7 minutes, 4° C.). The supernatant was decanted and stored in ice. The pellet was resuspended in 0.5 mL homogenization buffer, then homogenized and centrifuged as before. The supernatant was decanted, combined with the supernatant from the first centrifugation to form the S1 fraction, and centrifuged (200,000×gₘₐₓ, 30 minutes, 4° C.). The soluble (S2) fraction was decanted from the pellet (P2) fraction. The P2 pellet was resuspended in 0.5 mL of extraction buffer (homogenization buffer supplemented with 75 mM NaCl, 75 mM KCl, and 16 mM (1%, v/v) Triton X-100), homogenized and left in ice. After 20 minutes, the suspended P2 pellet was spun in an ultracentrifuge (200,000×gₘₐₓ, 20 minutes, at 4° C.). The Triton X-100-soluble P2 extract was decanted, aliquoted into storage tubes, snap frozen in liquid nitrogen, and stored at −80° C. The protein concentration was determined as described above.

Optimization of Rabbit Phospholipase C-γl Antibody Concentrations

Biotinylated goat anti-rabbit IgG at 1.0 μg of antibody/100 μL of PBS per well was coated onto streptavidin coated microtiter wells, as recommended by the manufacturer. Wells were incubated overnight at 4° C., then washed three times (5 minutes per wash) with PBS at room temperature. Rabbit anti-phospholipase C-γl antibody was serially diluted in PBS from a stock solution of 10 μg/mL to a final dilution of 0.31 μg/mL, and a volume of 100 μL of each solution was incubated with the biotinylated goat anti-rabbit IgG-coated streptavidin plates overnight at 4° C. The strips were washed three times (5 minutes per wash) with PBS at room temperature. Rat brain S1 fraction was loaded at 20 μg/100 μL PBS per well and incubated overnight at 4° C. Control wells contained normal rabbit IgG coated at 1.0 μg/100 μL per well and received rat brain S1 fraction (20 μg 100 μL). Unbound proteins were removed by washing the wells three times (5 minutes per wash) with 1.25× phospholipase C assay buffer (see below) at room temperature. Phospholipase C activity was then quantified as described below.

Stability Studies of Phospholipase C-γl Enzyme

Phospholipase C-γl was captured from a volume of 100 μL of a 200 μg/mL solution of rat brain S1 preparation using anti-phospholipase C-γl immobilized with biotin conjugated anti-rabbit immunoglobulin on streptavidin-coated microtiter plates, as described above. The wells were incubated with phospholipase C substrate, as described below, for the following times: 15, 30, 45 or 60 minutes. At the indicated time, the enzyme activity was quantified as described below.

Optimization of the Amount of Tissue Curve

Biotinylated anti-rabbit immunoglobulin was immobilized onto streptavidin coated wells as described above. A volume of 100 μL/well of anti-phospholipase C-γl antibody, at a concentration of 2.0 μg/mL of PBS, was then coated onto the immobilized biotin conjugated anti-rabbit immunoglobulin and left for 18 hours at 4° C., at which time the strips were washed three times (5 minutes each) with PBS at room temperature. Wells were incubated with hippocampal P2 extract: 0-100 μg of protein diluted to 100 μL in PBS per well. Control wells were coated with a volume of 100 μL normal rabbit IgG at 2.0 μg/mL of PBS, and received 100 μg of hippocampal P2 protein per 100 μL of PBS. All wells were incubated overnight at 4° C. Immobilized enzyme was assayed as described below.

Effect of Tyrosine Kinase Inhibitor on Phospholipase C-γl Catalytic Activity.

Anti-phospholipase C-γl antibodies were coated onto biotinylated anti-globulin immobilized on streptavidin at 200 ng of rabbit polyclonal antibody/100 μL per well. Each well was then incubated with 20 μg of rat hippocampal P2 extract overnight, and, subsequently, washed three times (5 minutes each) with room temperature PBS buffer. Wells were then incubated (20 minutes, 35° C.) in the presence of one of the following four solutions: Assay Dilution Buffer 1 (20 mM [3-(N-Morpholino)propanesulfonic acid], pH 7.2, 25 mM β-glycerol phosphate, 5 mM EGTA, 1 mM sodium orthovanadate, a phosphatase inhibitor, 1 mM dithiothreitol); 100 μM ATP, 15 mM MgCl₂ in Assay Dilution Buffer I; 0.5% (v/v) dimethyl sulfoxide in Assay Dilution Buffer I; or 50 μM genistein, a tyrosine kinase inhibitor, in Assay Dilution Buffer I containing 0.5% (v/v) dimethyl sulfoxide. The wells were rinsed three times (5 minutes each) in room temperature 1.25× phospholipase C assay buffer, and assayed for enzyme activity as described below.

Phospholipase C-γl Enzyme Activity Assay

Phospholipase C-γl enzyme activity was quantified essentially as described in Weeber et al. (Neurobiology of Learning and Memory 76 (2001) 151-182). Briefly, phospholipase C-γl enzyme was affinity purified from rat brain extracts and washed with 1.25× phospholipase C assay buffer (final assay concentration: 35 mM sodium phosphate, pH 6.8, 70 mM KCl, 0.8 mM EGTA, 0.8 mM CaCl₂), as described above. One-hundred microliters of 1.25× phospholipase C assay buffer was then added to each well, and the well was incubated for 5 minutes at 37° C. prior to adding the enzyme substrate. Twenty-five μL of [³H]PtdIns(4,5)P₂/Triton X-100 solution (final assay concentration: 0.200 mM PtdIns(4,5)P₂, 10,000-15,000 cpm/nmole, and 0.32 mM (0.02%, v/v) Triton X-100) was added and the incubation was continued for 30 minutes (unless otherwise noted). At the end of the reaction, 100 μL was removed from each well and transferred into tubes containing 125 μL of 1.0% (w/v) bovine serum albumin. Proteins and lipids were precipitated with 300 μL of ice-cold 10% (v/v) trichloroacetic acid and centrifuged at room temperature (14,000×gₘₐₓ for 4 minutes). 300 μL of the supernatant containing the reaction product ([³H]Ins(1,4,5)P₃) was removed and quantified by liquid scintillation spectroscopy. Immune-complex-dependent activity was calculated by subtracting background [³H]Ins(1,4,5)P₃ (release present in normal rabbit IgG antibody control samples) from the activity measured in wells containing anti-phospholipase C-γl antibody. Data were calculated as nanomoles Ins(1,4,5)P₃ product formed per minute, or per mg protein, or per minute per mg protein present in the extract from which the enzyme was affinity purified.

Results and Discussion

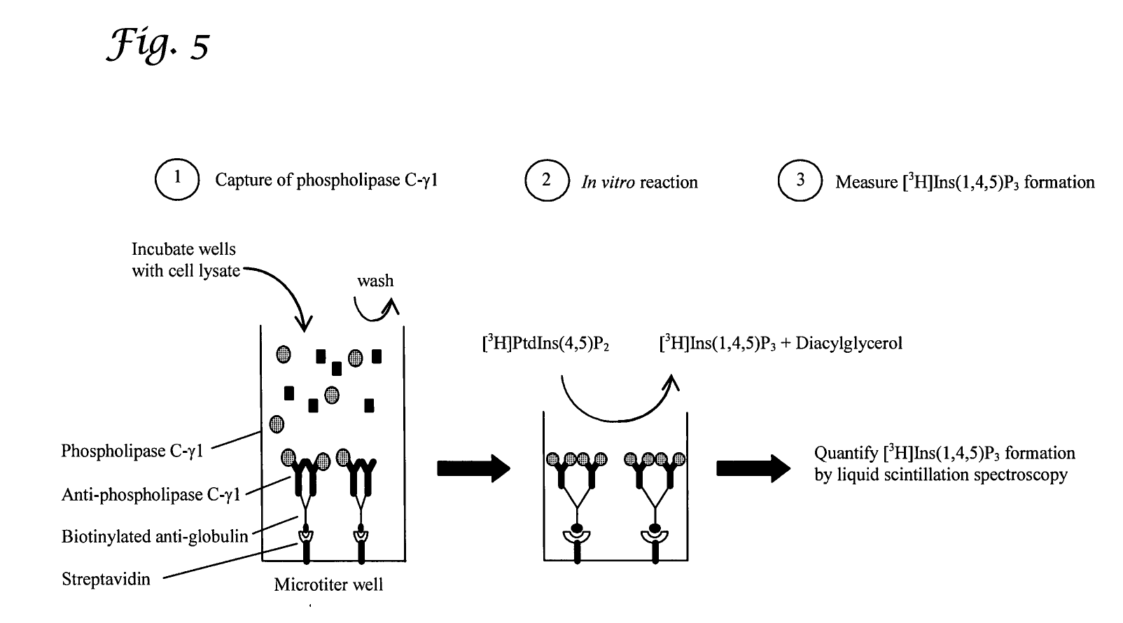

FIG. 5 depicts the experimental design used in these studies. Biotinylated anti-rabbit IgG was bound to streptavidin coated microtiter plate wells, creating a solid phase for immobilizing rabbit polyclonal antibodies against phospholipase C-γl. The immobilized anti-phospholipase C-γl antibodies were used to capture enzyme from rat brain tissue samples. Enzyme activity was determined using a conventional method to measure PtdIns(4,5)P₂ hydrolysis. The procedure consists of three steps: (i) phospholipase C-γl is captured from the cellular lysate using rabbit anti-phospholipase C-γl IgG, which is bound by biotinylated goat anti-rabbit IgG immobilized onto streptavidin coated microtiter plates, (ii) an in vitro reaction in which the lipase substrate, [³H]PtdIns(4,5)P₂, is hydrolyzed into [³H]Ins (1,4,5)P₃ and 1,2-diacylglycerol, and (iii) the [³H]Ins (1,4,5)P₃ product is quantified as a measure of phospholipase C enzyme activity using liquid scintillation spectroscopy.

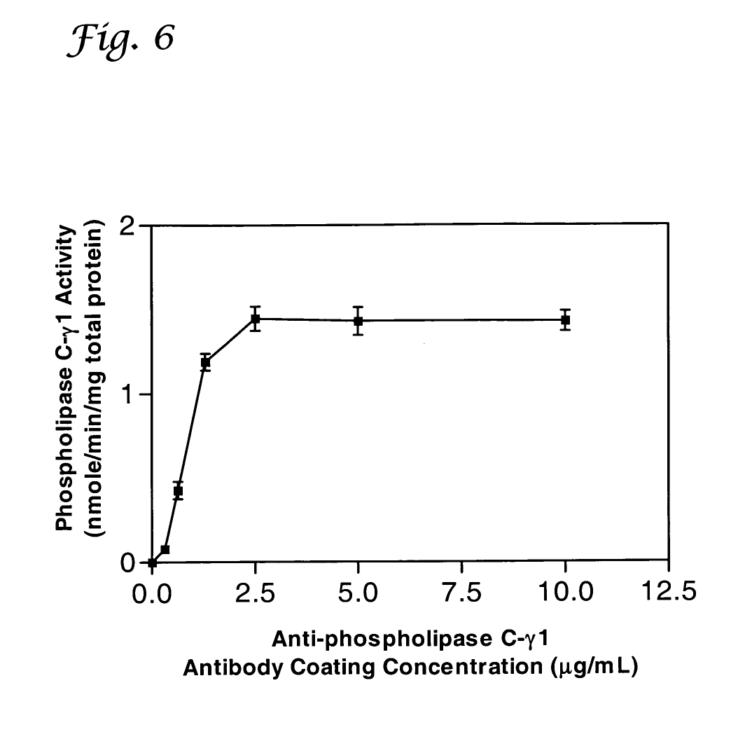

The optimal dilution of the rabbit anti-phospholipase C-γl antibody was determined by serial dilution of the antibody between 10.0 μg/mL and 0.31 μg/mL of PBS (FIG. 6). Streptavidin coated microtiter wells were first coated with 1.0 μg of biotinylated goat anti-rabbit IgG in 100 μL PBS and then coated with rabbit anti-phospholipase C-γl antibody in a dilution series starting at 10.0 μg/mL and ending at 0.31 μg/mL. The wells were rinsed with PBS, then incubated (18 hours, 4° C.) with 20 μg of rat brain S1 fraction diluted in 100 μL of PBS. After the incubation, the wells were rinsed and phospholipase C activity was measured as described in the Materials and Methods section. The reaction mix was incubated for 30 minutes. Each point is the average (n=3), after background subtraction. Some error bars are obstructed by symbols for the points.

Subsequent measurements of phospholipase C activity revealed increased formation of the reaction product, [³H]Ins(1,4,5)P₃, with increasing antibody amounts from 31 ng to 125 ng antibody per well, reaching a saturating plateau at antibody amounts greater than 125 ng per well. Therefore, the optimal coating concentration of antibody is approximately 1.25 μg/mL. In future assays, we coated the wells with 100 μL of a 2.0 μg/mL solution of rabbit phospholipase C-γl antibody per well. This concentration of antibody was used in order to saturate efficiently the binding sites on the wells and to avoid creating transient monolayers of antibody that can occur at higher concentrations of antibody due to non-specific protein-protein interactions.

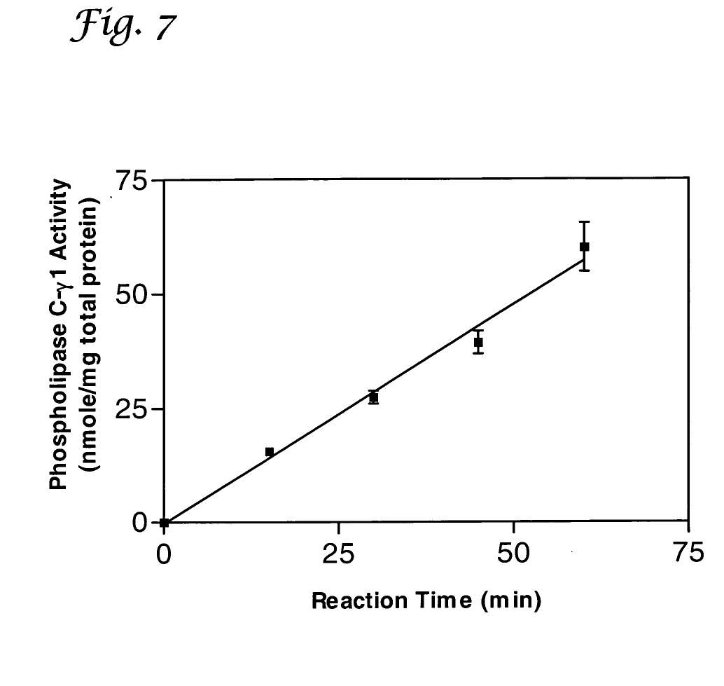

The stability of the immobilized phospholipase C-γl over time (FIG. 7) was determined by measuring enzyme activity associated with a constant amount of immobilized phospholipase C-γl captured from a 100 μL volume of rat brain S1 (200 g/mL), for varying periods of time ranging from 0 to 60 minutes. Streptavidin coated microtiter wells were coated with 1.0 μg biotinylated goat anti-rabbit IgG followed by coating with 0.2 μg rabbit anti-phospholipase C-γl antibody. Phospholipase C-γl was captured from 20 μg of rat brain S1 fraction. Phospholipase C activity was measured as described in the Materials and Methods section. Reactions were incubated for the following times: 15, 30, 45, and 60 minutes. Each point is the average (n=3), after background subtraction. Some error bars are obstructed by symbols for the points. The results demonstrate that the hydrolysis of substrate increased in a linear fashion for 60 minutes, demonstrating that immobilized phospholipase C-γl is stable for at least this length of incubation. Subsequent studies were performed employing a 30-minute incubation due to the ease of product detection at this time.

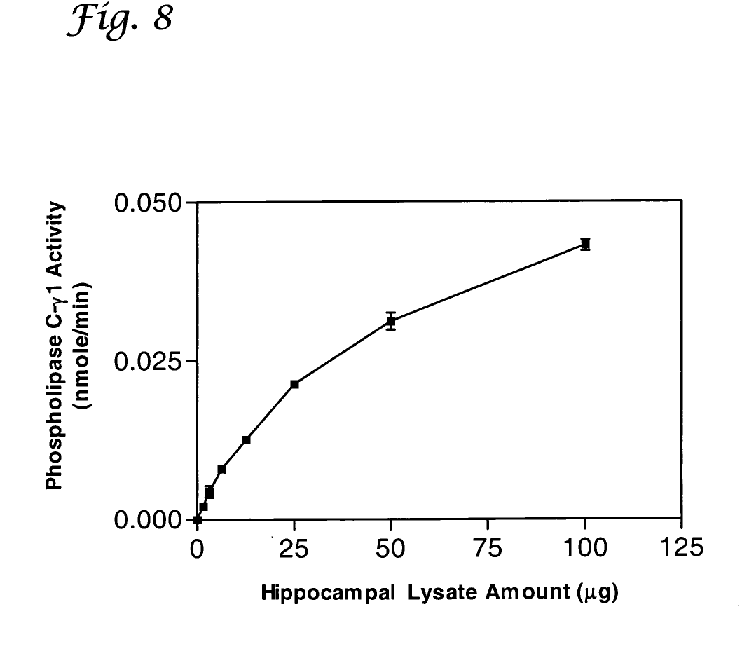

After the optimization of the immunoassay parameters, the linearity of the assay with increasing protein amounts was determined. Phospholipase C-γl was captured from protein amounts ranging from 1.56 μg-100 μg of rat hippocampal P2 fraction. Unbound proteins were rinsed from the wells and phospholipase C activity was measured as described in the Materials and Methods section. The reaction was incubated for 30 minutes. Each point is the average (n=3), after background subtraction. Some error bars are obstructed by symbols for the points. The results (FIG. 8) demonstrate a linear trend in phospholipase C-γl enzyme activity between 2 μg and approximately 25 μg of hippocampal formation P2 extract protein. Although the phospholipase C-γl enzyme activity curve fails to reach a plateau, it appears to be tending towards saturation. Higher tissue concentrations were not tested since it is not practical to use tissue samples in that range.

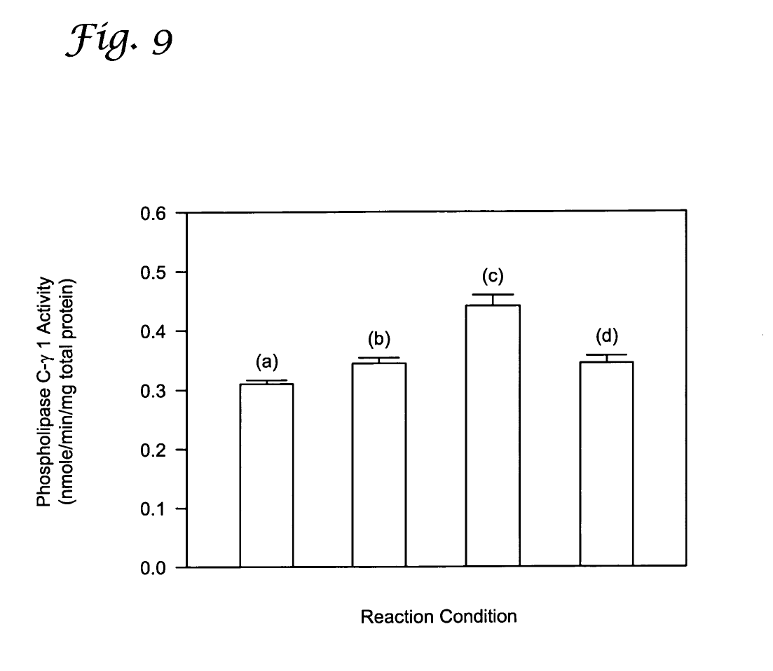

We sought to determine whether the technique that we used for the affinity capture of phospholipase C-γl allowed for the detection of tyrosine kinase(s) that associate with the phospholipase C-γl. To do this, we affinity captured phospholipase C-γl and incubated immune-complexes under conditions that allowed for substrate phosphorylation (i.e., in the presence of Mg²⁺ and ATP), or not. The wells were then rinsed with 1.25× phospholipase C assay buffer and the catalytic activity of the isozyme was determined. Incubation of anti-phospholipase C-γl immune-complexes with Mg²⁺-ATP increased phospholipase C enzyme activity (FIG. 9). Phospholipase C-γl captured by anti-phospholipase C-γl antibody was treated with or without ATP and/or genistein and phospholipase C activity was subsequently determined as described in the Materials and Methods section. Designations of the columns are as follows: (a) buffer without genistein or ATP; (b) buffer with genistein minus ATP; (c) buffer plus ATP minus genistein; (d) buffer with ATP and genistein. Each point is the average ±S.E.M. (n=7), after background subtraction. In order to determine the type of protein kinase responsible for the stimulation, we employed specific protein kinase inhibitors. In the presence of selective inhibitors of Ca²⁺-calmodulin-dependent protein kinase II, protein kinase C, and protein kinase A, ATP-dependent stimulation of phospholipase C-γl catalytic activity was still observed (data not shown), whereas the tyrosine kinase inhibitor, genistein, in the presence of Ca²⁺-calmodulin-dependent protein kinase II, protein kinase C, and protein kinase A inhibitors, completely inhibited the ATP-dependent stimulation of phospholipase C-γl catalytic activity. These results demonstrate that a tyrosine protein kinase co-purifies with immunoseparated phospholipase C-γl, and the effect of this kinase on phospholipase C-γl catalytic activity can be blocked using a specific tyrosine protein kinase inhibitor.

These studies have described the development of an immunochemical immobilization method for enzymes. This nonadsorbent, noncovalent, microtiter plate assay offers several advantages over solid phase immunoassays in which the capture antibody is coated directly to the surface of a microtiter plate well, and nonadsorbent, noncovalent methods in which the capture antibody is first biotinylated and then immobilized onto microtiter plates coated with streptavidin.

First, we tried direct adsorption of the phospholipase C antibody onto NUNC-Immuno MaxiSorp™ 96 well plates under various conditions (e.g., various buffers, pH, temperature and antibody concentrations) and we were not able to detect phospholipase C enzyme activity under any of the conditions we used. We, therefore, concluded that the most likely explanation for this result was that the antibody had become denatured by the surface, so that it was unable to bind phospholipase C. Subsequently, when we used the solid phase support described in this report, a catalytically active form of the enzyme was captured in the well. For the application of studying phospholipase C enzyme activity, the use of biotinylated anti-globulin to immobilize anti-phospholipase C antibodies was better than direct adsorption of anti-phospholipase C antibody onto plastic. Therefore, this technique allows for antibodies that bind poorly to plastic, or become denatured by plastic surfaces, to be of use in the assay.

Second, the technique described herein offers an advantage over the direct absorption of anti-globulin onto the plastic surface of a microtiter plate by removing the anti-globulin from direct contact with the hydrophobic surface using a streptavidin-biotin bridge. This linkage is hydrophilic in nature and has been shown to greatly increase the reactivity of antibodies bound to the surface of a microtiter plate (Suter et al., Immunol. Lett. 13 (1986) 313-316; Peterman et al., J. Immunol. Methods. 111 (1988) 271-275). Third, this method avoids biotinylation of the capture antibody, which is both time consuming and requires using materials that are known carcinogens.

In conclusion, this novel microtiter plate assay allows for the rapid capture and determination of catalytic activity of enzyme isoforms from tissue or cellular homogenates. It is technically simple to perform and could be employed in the study of any enzyme or protein or other biological molecule for which affinity capture antibodies are available.

The complete disclosures of all patents, patent applications including provisional patent applications, and publications, and electronically available material (e.g., GenBank amino acid and nucleotide sequence submissions) cited herein are incorporated by reference. The foregoing detailed description and examples have been provided for clarity of understanding only. No unnecessary limitations are to be understood therefrom. The invention is not limited to the exact details shown and described; many variations will be apparent to one skilled in the art and are intended to be included within the invention defined by the claims.

1. A multimolecular complex comprising:
an anti-immunoglobulin (anti-Ig) linking antibody reversibly bound to a substrate; and
a primary antibody bound to the anti-Ig linking antibody to yield an immobilized primary antibody.

2. The multimolecular complex of claim 1 wherein the substrate comprises streptavidin or biotin, and the anti-Ig linking antibody is reversibly bound to the substrate via a streptavidin-biotin interaction.

3. The multimolecular complex of claim 1 or 2 wherein the immobilized primary antibody is bound to the anti-Ig linking antibody through a binding interaction between the variable region of the anti-Ig linking antibody and the constant region of the primary antibody.

4. The multimolecular complex of claim 1 or 2 further comprising an antigen bound to the immobilized primary antibody.

5. The multimolecular complex of claim 4 wherein the bound antigen is a biomolecule.

6. The multimolecular complex of claim 5 wherein the bound biomolecule is an enzyme.

7. The multimolecular complex of claim 6 wherein the bound enzyme is biologically active.

8. The multimolecular complex of claims 1 or 2 wherein the bound antigen is a cell or a protein.

9. The multimolecular complex of claim 8 further comprising a second primary antibody, wherein the second primary antibody is bound to a site on the cell or protein that is different from the site bound to the immobilized primary antibody.

10. The multimolecular complex of claim 1 or 2 wherein the linking antibody comprises a modified anti-immunoglobulin antibody comprising a biotinylated F_{AB} fragment.

11. The multimolecular complex of claim 1 or 2 wherein the primary antibody comprises a modified antibody comprising a fusion between an F_{C} fragment and a receptor.

12. The multimolecular complex of claim 4 wherein the antigen comprises a receptor ligand.

13. A method for immobilizing an antigen comprising:
providing a multimolecular complex according to claim 1 or 2; and
contacting the multimolecular complex with a sample comprising an antigen, to yield a multimolecular complex comprising an antigen bound to the immobilized primary antibody.

14. The method of claim 13 further comprising disrupting the binding interaction between the immobilized primary antibody and the anti-Ig linking antibody so as to dissociate the primary antibody from the anti-Ig linking antibody.

15. The method of claim 13 further comprising disrupting the binding interaction between the antigen and the primary antibody so as to dissociate the antigen from the primary antibody.

16. The method of claim 13 further comprising detecting the presence of the bound antigen.

17. The method of claim 13 further comprising quantifying the amount of the bound antigen.

18. The method of claim 13 wherein the bound antigen is an enzyme, the method further comprising assaying the bound enzyme for activity.

19. The method of claim 13 wherein the bound antigen is a biologically active enzyme.

20. The method of claim 19 further comprising contacting the bound enzyme with an enzyme substrate to cause an enzymatic reaction.

21. The method of claim 13 wherein the bound antigen is a cell or a protein.

22. The method of claim 21 further comprising contacting the bound cell or protein with a second primary antibody, wherein the second primary antibody binds to a site on the cell or protein that is different from the site bound to the immobilized primary antibody.

23. The method of claim 21 further comprising sorting or purifying the cell or protein.

24. The method of claim 13 wherein the sample is a biological sample, an environmental sample, a food sample, or a cosmetic sample.

25. The method of claim 13 wherein the linking antibody comprises a modified anti-immunoglobulin antibody comprising a biotinylated F_{AB} fragment.

26. A method for detecting the presence of an antigen in a sample comprising:
providing a multimolecular complex according to claim 1 or 2;
contacting the multimolecular complex with a sample comprising an antigen, to yield a multimolecular complex comprising an antigen bound to the immobilized primary antibody; and
detecting the presence of the bound antigen.

27. The method of claim 26 wherein detecting the presence of the bound antigen comprises detecting the antigen immunologically using a sandwich assay.

28. The method of claim 27 wherein the sandwich assay comprises an enzyme linked immunosorbant assay (ELISA).

29. The method of claim 28 wherein the bound antigen is contacted with a second primary antibody that binds an epitope on the bound antigen that differs from the epitope bound by the immobilized primary antibody.

30. The method of claim 29 wherein the second primary antibody is detectably labeled.

31. The method of claim 26 wherein detecting the presence of the bound antigen comprises using immunologic, spectroscopic, thermodynamic or kinetic methods.

32. The method of claim 26 wherein the bound antigen is an enzyme, and wherein detecting the presence of the bound enzyme comprises assaying the enzyme for biological activity.

33. The method of claim 26 wherein the bound antigen is an enzyme, and the presence or absence of the bound enzyme is detected using an activity assay and an immunological assay; wherein success in detecting the bound enzyme immunologically combined with failure to detect bound enzyme activity is indicative of the binding of a non-active form of the enzyme to the immobilized primary antibody.

34. The method of claim 26 wherein the antigen is a contaminant, and wherein the method is performed for quality control purposes.

35. The method of claim 26 wherein the sample is biological sample, an environmental sample, a food sample, a cosmetic sample or a pharmaceutical sample.

36. The method of claim 26 wherein the linking antibody comprises a modified anti-immunoglobulin antibody comprising a biotinylated F_{AB} fragment

37. A method for detecting the presence of receptor ligand in a sample comprising:
providing a multimolecular complex comprising:
an anti-immunoglobulin (anti-Ig) linking antibody reversibly bound to a substrate; and
a primary antibody bound to the anti-Ig linking antibody to yield an immobilized primary antibody, wherein immobilized primary antibody comprises a modified antibody comprising a fusion between an F_{C} fragment and a receptor;
contacting the multimolecular complex with a sample comprising a receptor ligand to yield a multimolecular complex comprising a receptor ligand bound to the modified immobilized primary antibody; and
detecting the presence of the bound receptor ligand.

38. The method of claim 37 wherein the substrate comprises streptavidin or biotin, and the anti-Ig linking antibody is reversibly bound to the substrate via a streptavidin-biotin interaction.

39. The method of claim 37 further comprising removing the receptor ligand from solution.
